SEQUENCE LISTING (26 sequences)

SEQ ID NO: 1 - protein, 32 aa, Unknown
  Sequence of salmon calcitonin
CSNLSTCVLGKLSQELHKLQTYPRTNTGSGTP

SEQ ID NO: 2 - protein, 32 aa, Unknown
  Sequence of eel calcitonin
CSNLSTCVLGKLSQELHKLQTYPRTDVGAGTP

SEQ ID NO: 3 - protein, 32 aa, Unknown
  Sequence of chicken calcitonin
CASLSTCVLGKLSQELHKLQTYPRTDVGAGTP

SEQ ID NO: 4 - protein, 32 aa, Unknown
  Sequence of mouse calcitonin
CGNLSTCMLGTYTQDLNKFHTFPQTSIGVEAP

SEQ ID NO: 5 - protein, 32 aa, Unknown
  Sequence of rat calcitonin
CGNLSTCMLGTYTQDLNKFHTFPQTSIGVGAP

SEQ ID NO: 6 - protein, 32 aa, Unknown
  Sequence of horse calcitonin
CSNLSTCVLGTYTQDLNKFHTFPQTAIGVGAP

SEQ ID NO: 7 - protein, 32 aa, Unknown
  Sequence of canine-1 calcitonin
CSNLSTCVLGTYSKDLNNFHTFSGIGFGAETP

SEQ ID NO: 8 - protein, 32 aa, Unknown; identical to SEQ ID NO: 6
  Sequence of canine-2 calcitonin

SEQ ID NO: 9 - protein, 32 aa, Unknown
  Sequence of porcine calcitonin
CSNLSTCVLSAYWRNLNNFHRFSGMGFGPETP

SEQ ID NO: 10 - protein, 32 aa, Homo sapiens
  Sequence of human calcitonin
CGNLSTCMLGTYTQDFNKFHTFPQTAIGVGAP

SEQ ID NO: 11 - protein, 32 aa, Artificial Sequence
  Calcitonin mimetic peptide KBP-046
CGNLSTCMLGTYTQDFHRLQTYPKTDVGANAP

SEQ ID NO: 12 - protein, 32 aa, Artificial Sequence
  Calcitonin mimetic peptide KBP-047
CGNLSTCMLGRLSQDLNKFHTFPKTDVGANAP

SEQ ID NO: 13 - protein, 32 aa, Artificial Sequence
  Calcitonin mimetic peptide KBP-048
CGNLSTCMLGTYTQDFNKFHTFPKTDVGANAP

SEQ ID NO: 14 - protein, 32 aa, Artificial Sequence
  Calcitonin mimetic peptide KBP-049
CSNLSTCMLGRLSQDLNKFHTFPQTAIGVGAP

SEQ ID NO: 15 - protein, 32 aa, Artificial Sequence
  Calcitonin mimetic peptide KBP-050
CGNLSTCMLGRLSQDLHRLQTYPKTAIGVGAP

SEQ ID NO: 16 - protein, 32 aa, Artificial Sequence
  Calcitonin mimetic peptide KBP-051
CSNLSTCMLGRLSQDLHRLQTYPKTAIGVGAP

SEQ ID NO: 17 - protein, 32 aa, Artificial Sequence
  Calcitonin mimetic peptide KBP-052
CSNLSTCMLGTYTQDFHRLQTYPKTAIGVGAP

SEQ ID NO: 18 - protein, 32 aa, Artificial Sequence
  Calcitonin mimetic peptide KBP-053
CGNLSTCMLGRLTQDLHKLQTFPKTDVGANAP

SEQ ID NO: 19 - protein, 32 aa, Artificial Sequence
  Calcitonin mimetic peptide KBP-054
CGNLSTCMLGRYTQDFHKFQTFPKTDVGANAP

SEQ ID NO: 20 - protein, 32 aa, Artificial Sequence
  Calcitonin mimetic peptide KBP-058
CGNLSTCMLGRLTQDFHKLHTFPKTDVGANAP

SEQ ID NO: 21 - protein, 32 aa, Artificial Sequence
  Calcitonin mimetic peptide KBP-059
CGNLSTCMLGTYTQDLHKLHTFPKTDVGANAP

SEQ ID NO: 22 - protein, 32 aa, Artificial Sequence
  Calcitonin mimetic peptide KBP-060
CGNLSTCMLGTYTQDFHKLQTFPKTDVGANAP

SEQ ID NO: 23 - protein, 32 aa, Artificial Sequence
  Calcitonin mimetic peptide KBP-061
CGNLSTCMLGTYTQDFHRLQTFPQTDVGANAP

SEQ ID NO: 24 - protein, 32 aa, Artificial Sequence
  Calcitonin mimetic peptide KBP-062
CGNLSTCMLGRLTQDLNKFHTFPKTDVGANAP

SEQ ID NO: 25 - protein, 32 aa, Artificial Sequence
  Calcitonin mimetic peptide KBP-063
CGNLSTCMLGRLSQDLNKFHTFPQTDVGANAP

SEQ ID NO: 26 - protein, 32 aa, Artificial Sequence
  Generic calcitonin mimetic peptide sequence
  SITE 8: Xaa is Met or Val
  SITE 13: Xaa is Thr or Ser
  SITE 16: Xaa is Phe or Leu
  SITE 17: Xaa is Asn or His
  SITE 19: Xaa is Phe or Leu
  SITE 20: Xaa is Gln or His
  SITE 24: Xaa is Gln or Lys
CGNLSTCXLGRLXQDXXKXXTFPXTDVGANAP

(19) United States
(12) Patent Application Publication
(10) Pub. No.: US 2020/0199190 A1
(43) Pub. Date: Jun. 25, 2020

(54) Calcitonin Mimetics for Treating Diseases and Disorders

(71) Applicant: KeyBioscience AG, Stans (CH)

(72) Inventors: Kim Henriksen, Hillerød (DK); Morten Karsdal, Kobenhavn Ø (DK); Kim V. Andreassen, Frederiksberg (DK)

(73) Assignee: KeyBioscience AG, Stans (CH)

(21) Appl. No.: 16/495,887

(22) PCT Filed: Mar. 21, 2018
(86) PCT No.: PCT/EP2018/057102

§ 371 Date: Sep. 20, 2019

(30) Foreign Application Priority Data
Mar. 21, 2017 (GB) 1704429.8

(51) Int. Cl. C07K 14/585; A61K 31/155; A61K 47/12; A61K 47/18

(52) U.S. Cl. CPC C07K 14/585; A61K 31/155; A61K 9/0019; A61K 47/18; A61K 47/12

(57) ABSTRACT

The present invention relates to humanised calcitonin mimetics and their use in treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake.

The present invention relates to mimetics of calcitonin, and extends to their use as medicaments in the treatment of various diseases and disorders, including, but not limited to diabetes (Type I and Type II), excess bodyweight, excessive food consumption and metabolic syndrome, non-alcoholic steatohepatitis (NASH), alcoholic and non-alcoholic fatty liver disease, the regulation of blood glucose levels, the regulation of response to glucose tolerance tests, the regulation of food intake, the treatment of osteoporosis and the treatment of osteoarthritis.

Worldwide, there are about 250 million diabetics and the number is projected to double in the next two decades. Over 90% of this population suffers from type 2 diabetes mellitus (T2DM). It is estimated that only 50-60% of persons affected with T2DM or in stages preceding overt T2DM are currently diagnosed.

T2DM is a heterogeneous disease characterized by abnormalities in carbohydrate and fat metabolism. The causes of T2DM are multi-factorial and include both genetic and environmental elements that affect β-cell function and insulin sensitivity in tissues such as muscle, liver, pancreas and adipose tissue. As a consequence impaired insulin secretion is observed and paralleled by a progressive decline in β-cell function and chronic insulin resistance. The inability of the endocrine pancreas to compensate for peripheral insulin resistance leads to hyperglycaemia and onset of clinical diabetes. Tissue resistance to insulin-mediated glucose uptake is now recognized as a major pathophysiologic determinant of T2DM.

A success criterion for an optimal T2DM intervention is the lowering of blood glucose levels, which can be both chronic lowering of blood glucose levels and increased ability to tolerate high glucose levels after food intake, described by lower peak glucose levels and faster clearance. Both of these situations exert less strain on β-cell insulin output and function.

Type I diabetes is characterised by a loss of the ability to produce insulin in response to food intake and hence an inability to regulate blood glucose to a normal physiological level.

The physical structure of bone may be compromised by a variety of factors, including disease and injury. One of the most common bone diseases is osteoporosis, which is characterized by low bone mass and structural deterioration of bone tissue, leading to bone fragility and an increased susceptibility to fractures, particularly of the hip, spine and wrist. Osteoporosis develops when there is an imbalance such that the rate of bone resorption exceeds the rate of bone formation. Administering an effective amount of an anti-resorptive agent, such as calcitonin, has shown to prevent resorption of bone.

Inflammatory or degenerative diseases, including diseases of the joints, e.g. osteoarthritis (OA), rheumatoid arthritis (RA) or juvenile rheumatoid arthritis (JRA), and including inflammation that results from autoimmune response, e.g. lupus, ankylosing spondylitis (AS) or multiple sclerosis (MS), can lead to substantial loss of mobility due to pain and joint destruction. Cartilage that covers and cushions bone within joints may become degraded over time thus undesirably permitting direct contact of two bones that can limit motion of one bone relative to the other and/or cause damage to one by the other during motion of the joint. Subchondral bone just beneath the cartilage may also degrade. Administering an effective amount of an anti-resorptive agent, such as calcitonin, may prevent resorption of bone.

Calcitonins are highly conserved over a wide range of species. Full-length native calcitonin is 32 amino acids in length. The sequences of examples of natural calcitonins are set out below:

(SEQ ID NO: 1)

| | |
|---|---|
| Salmon | CSNLSTCVLGKLSQELHKLQTYPRTNTGSGTP |

(SEQ ID NO: 2)

| | |
|---|---|
| Eel | CSNLSTCVLGKLSQELHKLQTYPRTDVGAGTP |

(SEQ ID NO: 3)

| | |
|---|---|
| Chicken | CASLSTCVLGKLSQELHKLQTYPRTDVGAGTP |

(SEQ ID NO: 4)

| | |
|---|---|
| Mouse | CGNLSTCMLGTYTQDLNKFHTFPQTSIGVEAP |

(SEQ ID NO: 5)

| | |
|---|---|
| Rat | CGNLSTCMLGTYTQDLNKFHTFPQTSIGVGAP |

(SEQ ID NO: 6)

| | |
|---|---|
| Horse | CSNLSTCVLGTYTQDLNKFHTFPQTAIGVGAP |

(SEQ ID NO: 7)

| | |
|---|---|
| Canine-1 | CSNLSTCVLGTYSKDLNNFHTFSGIGFGAETP |

(SEQ ID NO: 8)

| | |
|---|---|
| Canine-2 | CSNLSTCVLGTYTQDLNKFHTFPQTAIGVGAP |

(SEQ ID NO: 9)

| | |
|---|---|
| Porcine | CSNLSTCVLSAYWRNLNNFHRFSGMGFGPETP |

(SEQ ID NO: 10)

| | |
|---|---|
| Human | CGNLSTCMLGTYTQDFNKFHTFPQTAIGVGAP |

Synthetic variants of natural calcitonins having modified amino acid sequences which are intended to provide improved properties are disclosed in WO2013/067357 and WO 2015/071229.

There is a continuing need to develop calcitonin analogues having still further improved properties, or at least providing alternative artificial sequences improving on the properties of the naturally occurring calcitonins, particularly in respect of amylin and calcitonin receptor agonism, while eliminating CGRP-Receptor agonism, and thereby ensuring the optimal in vivo efficacy to safety ratio.

However, an issue with peptide driven therapeutic agents is the creation of anti-drug antibodies (ADAs) in response to host exposure of the given treatment (Wu B et al AAPS J. 2016 November; 18(6):1335-1350; Schellekens H, Clin Ther. 2002 November; 24(11):1720-40). Emergence of ADAs are even more common when using peptide therapies derived from other organisms such salmon calcitonin as a therapeutic agent in various bone related diseases (Grauer et al, Exp clin Endo Diabetes, 1995) and as exanatide to treat T2DM (Fineman M S et al, Diabetes Obes Metab. 2012 June; 14(6):546-54), which can interfere with the efficacy of the given treatment.

The teleost/avian calcitonins have a roughly 50% sequence homology with the endogenous ligand, human calcitonin (hCT), hence it would be advantageous to increase the sequence homology towards hCT in an effort to attenuate ADA production by mimicking an endogenous target while retaining the unique ligand properties of the teleost/avian group of calcitonins.

### SUMMARY OF THE INVENTION

The present invention now provides calcitonin mimetics which are highly similar to human calcitonin (by % identity) but which, surprisingly, also demonstrate useful therapeutic properties, such as improved appetite suppression. As shown herein, the development of such efficacious ‘humanised’ calcitonin mimetics is a far from trivial matter. Tables 1A and 1B (below) provides the amino acid sequences of calcitonin mimetics that were developed and tested by the Applicant. It was found that humanising certain residues (i.e. replacing certain residues with the equivalent residue found in human calcitonin) resulted in improvements in efficacy, whilst replacing others significantly reduced efficacy.

TABLE 1
Additional Sequence modifications: N-term acetylation, C-TERM AMIDATION
% hCT = % identity to human calciton; hCT = SEQ ID NO: 10

| Number | % hcT | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| hcT | 100 | C | G | N | L | S | T | C | M | L | G | T | Y | T | Q | D | F |
| KPB-046 | 69 | C | G | N | L | S | T | C | M | L | G | T | Y | T | Q | D | F |
| SEQ ID | | | | | | | | | | | | | | | | | |
| NO: 11 | | | | | | | | | | | | | | | | | |
| KPB-047 | 72 | C | G | N | L | S | T | C | M | L | G | R | L | S | Q | D | L |
| SEQ ID | | | | | | | | | | | | | | | | | |
| NO: 12 | | | | | | | | | | | | | | | | | |
| KPB-048 | 64 | C | G | N | L | S | T | C | M | L | G | T | Y | T | Q | D | F |
| SEQ ID | | | | | | | | | | | | | | | | | |
| NO: 13 | | | | | | | | | | | | | | | | | |
| KPB-049 | 64 | C | S | N | L | S | T | C | M | L | G | R | L | S | Q | D | L |
| SEQ ID | | | | | | | | | | | | | | | | | |
| NO: 14 | | | | | | | | | | | | | | | | | |
| KPB-050 | 69 | C | G | N | L | S | T | C | M | L | G | R | L | S | Q | D | L |
| SEQ ID | | | | | | | | | | | | | | | | | |
| NO: 15 | | | | | | | | | | | | | | | | | |
| KPB-051 | 66 | C | S | N | L | S | T | C | M | L | G | R | L | S | Q | D | L |
| SEQ ID | | | | | | | | | | | | | | | | | |
| NO: 16 | | | | | | | | | | | | | | | | | |
| KPB-052 | 78 | C | S | N | L | S | T | C | M | L | G | T | Y | T | Q | D | F |
| SEQ ID | | | | | | | | | | | | | | | | | |
| NO: 17 | | | | | | | | | | | | | | | | | |
| KPB-053 | 66 | C | G | N | L | S | T | C | M | L | G | R | L | T | Q | D | L |
| SEQ ID | | | | | | | | | | | | | | | | | |
| NO: 18 | | | | | | | | | | | | | | | | | |
| KPB-054 | 78 | C | G | N | L | S | T | | | L | G | R | Y | T | Q | D | F |
| SEQ ID | | | | | | | | | | | | | | | | | |
| NO: 19 | | | | | | | | | | | | | | | | | |
| KPB-058 | 72 | C | G | N | L | S | T | C | M | L | G | R | L | T | Q | D | F |
| SEQ ID | | | | | | | | | | | | | | | | | |
| NO: 20 | | | | | | | | | | | | | | | | | |
| KPB-059 | 75 | C | G | N | L | S | T | C | M | L | G | T | Y | T | Q | D | L |
| SEQ ID | | | | | | | | | | | | | | | | | |
| NO: 21 | | | | | | | | | | | | | | | | | |
| KPB-060 | 75 | C | G | N | L | S | T | C | M | L | G | T | Y | T | Q | D | F |
| SEQ ID | | | | | | | | | | | | | | | | | |
| NO: 22 | | | | | | | | | | | | | | | | | |
| KPB-061 | 75 | C | G | N | L | S | T | C | M | L | G | T | Y | T | Q | D | F |
| SEQ ID | | | | | | | | | | | | | | | | | |
| NO: 23 | | | | | | | | | | | | | | | | | |
| KPB-062 | 75 | C | G | N | L | S | T | C | M | L | G | R | L | T | Q | D | L |
| SEQ ID | | | | | | | | | | | | | | | | | |
| NO: 24 | | | | | | | | | | | | | | | | | |
| KPB-063 | 75 | C | G | N | L | S | T | C | M | L | G | R | L | S | Q | D | L |
| SEQ ID | | | | | | | | | | | | | | | | | |
| NO: 25 | | | | | | | | | | | | | | | | | |
| | Number | 17 | 18 | 19 | 20 | 21 | 22 | 23 | 24 | 25 | 26 | 27 | 28 | 29 | 30 | 31 | 32 |
| | hcT | N | K | F | N | T | F | P | Q | T | A | I | G | V | G | A | P |
| | KPB-046 | H | R | L | Q | T | Y | P | K | T | D | V | G | A | N | A | P |
| | SEQ ID | | | | | | | | | | | | | | | | |
| | NO: 11 | | | | | | | | | | | | | | | | |
| | KPB-047 | N | K | F | N | T | F | P | K | T | D | V | G | A | N | A | P |
| | SEQ ID | | | | | | | | | | | | | | | | |
| | NO: 12 | | | | | | | | | | | | | | | | |
| | KPB-048 | N | K | F | N | T | F | P | K | T | D | V | G | A | N | A | P |
| | SEQ ID | | | | | | | | | | | | | | | | |
| | NO: 13 | | | | | | | | | | | | | | | | |
| | KPB-049 | N | K | F | N | T | F | P | Q | T | A | I | G | V | G | A | P |
| | SEQ ID | | | | | | | | | | | | | | | | |
| | NO: 14 | | | | | | | | | | | | | | | | |
| | KPB-050 | H | R | L | Q | T | Y | P | K | T | A | I | G | V | G | A | P |
| | SEQ ID | | | | | | | | | | | | | | | | |
| | NO: 15 | | | | | | | | | | | | | | | | |
| | KPB-051 | H | R | L | Q | T | Y | P | K | T | A | I | G | V | G | A | P |
| | SEQ ID | | | | | | | | | | | | | | | | |
| | NO: 16 | | | | | | | | | | | | | | | | |
| | KPB-052 | H | R | L | Q | T | Y | P | K | T | A | I | G | V | G | A | P |
| | SEQ ID | | | | | | | | | | | | | | | | |
| | NO: 17 | | | | | | | | | | | | | | | | |
| | KPB-053 | K | K | L | Q | T | F | P | K | T | D | V | G | A | N | A | P |
| | SEQ ID | | | | | | | | | | | | | | | | |
| | NO: 18 | | | | | | | | | | | | | | | | |
| | KPB-054 | H | K | P | Q | T | F | P | K | T | D | V | G | A | N | A | P |
| | SEQ ID | | | | | | | | | | | | | | | | |
| | NO: 19 | | | | | | | | | | | | | | | | |
| | KPB-058 | H | K | L | K | T | F | P | K | T | D | V | G | A | N | A | P |
| | SEQ ID | | | | | | | | | | | | | | | | |
| | NO: 20 | | | | | | | | | | | | | | | | |
| | KPB-059 | H | K | L | H | T | F | P | K | T | D | V | G | A | N | A | P |
| | SEQ ID | | | | | | | | | | | | | | | | |
| | NO: 21 | | | | | | | | | | | | | | | | |
| | KPB-060 | H | R | L | Q | T | F | P | Q | T | D | V | G | A | N | A | P |
| | SEQ ID | | | | | | | | | | | | | | | | |
| | NO: 22 | | | | | | | | | | | | | | | | |
| | KPB-061 | H | R | L | Q | T | F | P | Q | T | D | V | G | A | N | A | P |
| | SEQ ID | | | | | | | | | | | | | | | | |
| | NO: 23 | | | | | | | | | | | | | | | | |
| | KPB-062 | N | K | F | R | T | F | P | K | T | D | V | G | A | N | A | P |
| | SEQ ID | | | | | | | | | | | | | | | | |
| | NO: 24 | | | | | | | | | | | | | | | | |
| | KPB-063 | N | K | F | K | T | F | P | Q | T | D | V | G | A | N | A | P |
| | SEQ ID | | | | | | | | | | | | | | | | |
| | NO: 25 | | | | | | | | | | | | | | | | |

Whilst most of the tested peptides produced some degree of appetite suppression over 4 hours, those peptides highlighted in Tables 1A and 1B (KBP-047 [SEQ ID NO: 12], -053 [SEQ ID NO: 18], -058 [SEQ ID NO: 20], -062 [SEQ ID NO: 24], and -063 [SEQ ID NO: 25]) produced a far greater reduction in food intake and were the only peptides shown to produce a sustained effect over 24 hours.

Accordingly, in a first aspect the present invention relates to a peptide that is a calcitonin mimetic, wherein the peptide is:

(SEQ ID NO: 26)
CGNLSTC [symbol] LGRL [symbol] QD [symbol] X4K [symbol] TFP [symbol] TDVGANAP

wherein

X₁=M or V

X₂=T or S

X₃=F or L

X₄=N or H,

X₅=F or L

X₆=Q or H

X₇=Q or K

Preferably, X₂ is T, X₃ is L, X₄ is N, X₅ is F, X₆ is H and/or X₇ is K.

Preferably, X₄ is N, X₅ is F, and X₆ is H.

Preferably, the peptide has an identity to human calcitonin of at least 65%, more preferably at least 70%, and most preferably at least 75%.

Preferably, the peptide is selected from one of the following:

(SEQ ID NO: 12)
KBP-047: CGNLSTCMLGRLSQDLNKFHTFPKTDVGANAP,
(SEQ ID NO: 18)
KBP-053: CGNLSTCMLGRLTQDLHKLQTFPKTDVGANAP,
(SEQ ID NO: 20)
KBP-058: CGNLSTCMLGRLTQDFHKLHTFPKTDVGANAP,
(SEQ ID NO: 24)
KBP-062: CGNLSTCMLGRLTQDLNKFHTFPKTDVGANAP,
or
(SEQ ID NO: 25)
KBP-063: CGNLSTCMLGRLSQDLNKFHTFPQTDVGANAP

The peptides of the invention may be acylated at its N-terminal or otherwise modified to reduce the positive charge of the first amino acid and independently of that may be amidated at its C-terminal.

The peptide may be formulated for administration as a pharmaceutical and may be formulated for enteral or parenteral administration. Preferred formulations are injectable, preferably for subcutaneous injection, however the peptide may be formulated with a carrier for oral administration, and optionally wherein the carrier increases the oral bioavailability of the peptide. Suitable carriers include ones that comprise 5-CNAC, SNAD, or SNAC.

Optionally, the peptide is formulated in a pharmaceutical composition for oral administration comprising coated citric acid particles, and wherein the coated citric acid particles increase the oral bioavailability of the peptide.

The invention includes a peptide of the invention for use as a medicament. The peptide may be for use in treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake. In particular, the peptides may be used to lower an undesirably high fasting blood glucose level or to lower an undesirably high HbA1c or to reduce an undesirably high response to a glucose tolerance test.

In some embodiments, the N-terminal side of the calcitonin mimetics discussed supra is modified to reduce the positive charge of the first amino acid. For example, an acetyl, propionyl, or succinyl group may be substituted on cysteine-1. Alternative ways of reducing positive charge include, but are not limited to, polyethylene glycol-based PEGylation, or the addition of another amino acid such as glutamic acid or aspartic acid at the N-terminus. Alternatively, other amino acids may be added to the N-terminus of peptides discussed supra including, but not limited to, lysine, glycine, formylglycine, leucine, alanine, acetyl alanine, and dialanyl. As those of skill in the art will appreciate, peptides having a plurality of cysteine residues frequently form a disulfide bridge between two such cysteine residues. All such peptides set forth herein are defined as optionally including one or more such disulphide bridges, particularly at the Cys1-Cys7 locations. Mimicking this, the cysteines at positions 1 and 7 may jointly be replaced by an α-aminosuberic acid linkage.

While calcitonin mimetics of the present disclosure may exist in free acid form, it is preferred that the C-terminal amino acid be amidated. Applicants expect that such amidation may contribute to the effectiveness and/or bioavailability of the peptide. A preferred technique for manufacturing amidated versions of the calcitonin mimetics of the present disclosure is to react precursors (having glycine in place of the C-terminal amino group of the desired amidated product) in the presence of peptidylglycine alpha-amidating monooxygenase in accordance with known techniques wherein the precursors are converted to amidated products in reactions described, for example, in U.S. Pat. No. 4,708,934 and EP0308067 and EP0382403.

Production of amidated products may also be accomplished using the process and amidating enzyme set forth by Consalvo, et al in U.S. Pat. No. 7,445,911; Miller et al, US2006/0292672; Ray et al, 2002, Protein Expression and Purification, 26:249-259; and Mehta, 2004, Biopharm. International, July, pp. 44-46.

The production of the preferred amidated peptides may proceed, for example, by producing glycine-extended precursor in E. coli as a soluble fusion protein with glutathione-S-transferase, or by direct expression of the precursor in accordance with the technique described in U.S. Pat. No. 6,103,495. Such a glycine extended precursor has a molecular structure that is identical to the desired amidated product except at the C-terminus (where the product terminates —X—NH₂, while the precursor terminates —X-gly, X being the C-terminal amino acid residue of the product). An alpha-amidating enzyme described in the publications above catalyzes conversion of precursors to product. That enzyme is preferably recombinantly produced, for example, in Chinese Hamster Ovary (CHO) cells), as described in the Biotechnology and Biopharm. articles cited above.

Free acid forms of peptide active agents of the present disclosure may be produced in like manner, except without including a C-terminal glycine on the “precursor”, which precursor is instead the final peptide product and does not require the amidation step.

Except where otherwise stated, the preferred dosage of the calcitonin mimetics of the present disclosure is identical for both therapeutic and prophylactic purposes. Desired dosages are discussed in more detail, infra, and differ depending on mode of administration.

Except where otherwise noted or where apparent from context, dosages herein refer to weight of active compounds (i.e. calcitonin mimetics) unaffected by or discounting pharmaceutical excipients, diluents, carriers or other ingredients, although such additional ingredients are desirably included. Any dosage form (capsule, tablet, injection or the like) commonly used in the pharmaceutical industry for delivery of peptide active agents is appropriate for use herein, and the terms “excipient”, “diluent”, or “carrier” includes such non-active ingredients as are typically included, together with active ingredients in such dosage form in the industry. A preferred oral dosage form is discussed in more detail, infra, but is not to be considered the exclusive mode of administering the active agents of the present disclosure.

The calcitonin mimetics of the present disclosure can be administered to a patient to treat a number of diseases or disorders. As used herein, the term “patient” means any organism belonging to the kingdom Animalia. In an embodiment, the term “patient” refers to vertebrates, more preferably, mammals including humans.

Accordingly, the present disclosure includes the use of the peptides in a method of treatment of type I diabetes, Type II diabetes or metabolic syndrome, obesity, or of appetite suppression, or for mitigating insulin resistance, or for reducing an undesirably high fasting serum glucose level, or for reducing an undesirably high peak serum glucose level, or for reducing an undesirably high peak serum insulin level, or for reducing an undesirably large response to a glucose tolerance test, or for treating osteoporosis, or for treating osteoarthritis, or for treating non-alcoholic steatohepatitis (NASH), or for treating alcoholic fatty liver disease.

There are a number of art-recognized measures of normal range for body weight in view of a number of factors such as gender, age and height. A patient in need of treatment or prevention regimens set forth herein include patients whose body weight exceeds recognized norms or who, due to heredity, environmental factors or other recognized risk factor, are at higher risk than the general population of becoming overweight or obese. In accordance with the present disclosure, it is contemplated that the calcitonin mimetics may be used to treat diabetes where weight control is an aspect of the treatment.

In an embodiment, the method includes enteral administration to a patient in need thereof for treatment of a said condition of a pharmaceutically effective amount of any one of the peptides described herein.

In an embodiment, the method includes parenteral administration to a patient in need thereof for treatment of a said condition of a pharmaceutically effective amount of any one of the peptides described herein. For parenteral administration (including intraperitoneal, subcutaneous, intravenous, intradermal or intramuscular injection), solutions of a peptide of the present disclosure in either sesame or peanut oil or in aqueous propylene glycol may be employed, for example. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Peptides may be formulated in sterile form in multiple or single dose formats such as being dispersed in a fluid carrier such as sterile physiological saline or 5% saline dextrose solutions commonly used with injectables.

Said method may include a preliminary step of determining whether the patient suffers from a said condition, and/or a subsequent step of determining to what extent said treatment is effective in mitigating the condition in said patient, e.g. in each case, carrying out an oral glucose tolerance test or a resting blood sugar level.

For improved control over the weight of the patient, to produce a loss of weight or an avoidance of weight gain, the active compound is preferably administered once daily or more such as at least twice per day, e.g. from 2-4 times per day. Formulations of the active compound may contain a unit dosage appropriate for such an administration schedule. The active compounds may be administered with a view to controlling the weight of a patient undergoing treatment for diabetes or metabolic syndrome.

Oral enteral formulations are for ingestion by swallowing for subsequent release in the intestine below the stomach, and hence delivery via the portal vein to the liver, as opposed to formulations to be held in the mouth to allow transfer to the bloodstream via the sublingual or buccal routes.

Suitable dosage forms for use in the present disclosure include tablets, mini-tablets, capsules, granules, pellets, powders, effervescent solids and chewable solid formulations. Such formulations may include gelatin which is preferably hydrolysed gelatin or low molecular weight gelatin. Such formulations may be obtainable by freeze drying a homogeneous aqueous solution comprising a calcitonin mimetic and hydrolysed gelatin or low molecular weight gelatin and further processing the resulting solid material into said oral pharmaceutical formulation, and wherein the gelatin may have a mean molecular weight from 1000 to 15000 Daltons. Such formulations may include a protective carrier compound such as 5-CNAC or others as disclosed herein.

Whilst oral formulations such as tablets and capsules are preferred, compositions for use in the present disclosure may take the form of syrups, elixirs or the like and suppositories or the like. Oral delivery is generally the delivery route of choice since it is convenient, relatively easy and generally painless, resulting in greater patient compliance relative to other modes of delivery. However, biological, chemical and physical barriers such as varying pH in the gastrointestinal tract, powerful digestive enzymes, and active agent impermeable gastrointestinal membranes, makes oral delivery of calcitonin like peptides to mammals problematic, e.g. the oral delivery of calcitonins, which are long-chain polypeptide hormones secreted by the parafollicular cells of the thyroid gland in mammals and by the ultimobranchial gland of birds and fish, originally proved difficult due, at least in part, to the insufficient stability of calcitonin in the gastrointestinal tract as well as the inability of calcitonin to be readily transported through the intestinal walls into the blood stream.

Suitable oral formulations are however described below.

### Treatment of Patients

In an embodiment, a calcitonin mimetic of the present disclosure is administered at adequate dosage to maintain serum levels of the mimetic in patients between 5 picograms and 500 nanograms per milliliter, preferably between 50 picograms and 250 nanograms, e.g. between 1 and 100 nanograms per milliliter. The serum levels may be measured by radioimmunoassay techniques known in the art. The attending physician may monitor patient response, and may then alter the dosage somewhat to account for individual patient metabolism and response. Near simultaneous release is best achieved by administering all components of the present disclosure as a single pill or capsule. However, the disclosure also includes, for example, dividing the required amount of the calcitonin mimetic among two or more tablets or capsules which may be administered together such that they together provide the necessary amount of all ingredients. “Pharmaceutical composition,” as used herein includes but is not limited to a complete dosage appropriate to a particular administration to a patient regardless of whether one or more tablets or capsules (or other dosage forms) are recommended at a given administration.

A calcitonin mimetic of the present disclosure may be formulated for oral administration using the methods employed in the Unigene Enteripep® products. These may include the methods as described in U.S. Pat. Nos. 5,912,014, 6,086,918, 6,673,574, 7,316,819, 8,093,207, and US Publication No. 2009/0317462. In particular, it may include the use of conjugation of the compound to a membrane translocator such as the protein transduction domain of the HIV TAT protein, co-formulation with one or more protease inhibitors, and/or a pH lowering agent which may be coated and/or an acid resistant protective vehicle and/or an absorption enhancer which may be a surfactant.

In an embodiment, a calcitonin mimetic of the present disclosure is preferably formulated for oral delivery in a manner known in U.S. Patent Publication No. 2009/0317462.

In an embodiment, a calcitonin mimetic of the present disclosure may be formulated for enteral, especially oral, administration by admixture with a suitable carrier compound. Suitable carrier compounds include those described in U.S. Pat. Nos. 5,773,647 and 5,866,536 and amongst these, 5-CNAC (N-(5-chlorosalicyloyl)-8-aminocaprylic acid, commonly as its disodium salt) is particularly effective. Other preferred carriers or delivery agents are SNAD (sodium salt of 10-(2-Hydroxybenzamido)decanoic acid) and SNAC (sodium salt of N-(8-[2-hydroxybenzoyl]amino)caprylic acid). In an embodiment, a pharmaceutical composition of the present disclosure comprises a delivery effective amount of carrier such as 5-CNAC, i.e. an amount sufficient to deliver the compound for the desired effect. Generally, the carrier such as 5-CNAC is present in an amount of 2.5% to 99.4% by weight, more preferably 25% to 50% by weight of the total composition.

In addition, WO 00/059863 discloses the disodium salts of formula I

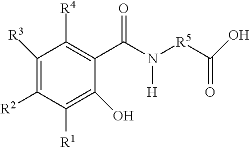

wherein
R¹, R², R³, and R⁴ are independently hydrogen, —OH, —NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl (C₁-C₁₂ alkylene); and R⁶ and R⁷ are independently hydrogen, oxygen, or C₁-C₄ alkyl; and hydrates and solvates thereof as particularly efficacious for the oral delivery of active agents, such as calcitonins, e.g. salmon calcitonin, and these may be used in the present disclosure.

Preferred enteric formulations using optionally micronised 5-CNAC may be generally as described in WO2005/014031.

The compound may be formulated for oral administration using the methods employed in the Capsitonin product of Bone Medical Limited. These may include the methods incorporated in Axcess formulations. More particularly, the active ingredient may be encapsulated in an enteric capsule capable of withstanding transit through the stomach. This may contain the active compound together with a hydrophilic aromatic alcohol absorption enhancer, for instance as described in WO02/028436. In a known manner the enteric coating may become permeable in a pH sensitive manner, e.g. at a pH of from 3 to 7. WO2004/091584 also describes suitable formulation methods using aromatic alcohol absorption enhancers.

The compound may be formulated using the methods seen in the Oramed products, which may include formulation with omega-3 fatty acid as seen in WO2007/029238 or as described in U.S. Pat. No. 5,102,666.

Generally, the pharmaceutically acceptable salts (especially mono or di sodium salts), solvates (e.g. alcohol solvates) and hydrates of these carriers or delivery agents may be used.

Oral administration of the pharmaceutical compositions according to the disclosure can be accomplished regularly, e.g. once or more on a daily or weekly basis; intermittently, e.g. irregularly during a day or week; or cyclically, e.g. regularly for a period of days or weeks followed by a period without administration. The dosage form of the pharmaceutical compositions of the presently disclosed embodiments can be any known form, e.g. liquid or solid dosage forms. The liquid dosage forms include solution emulsions, suspensions, syrups and elixirs. In addition to the active compound and carrier such as 5-CNAC, the liquid formulations may also include inert excipients commonly used in the art such as, solubilizing agents e.g. ethanol; oils such as cottonseed, castor and sesame oils; wetting agents; emulsifying agents; suspending agents; sweeteners; flavourings; and solvents such as water. The solid dosage forms include capsules, soft-gel capsules, tablets, caplets, powders, granules or other solid oral dosage forms, all of which can be prepared by methods well known in the art. The pharmaceutical compositions may additionally comprise additives in amounts customarily employed including, but not limited to, a pH adjuster, a preservative, a flavorant, a taste-masking agent, a fragrance, a humectant, a tonicifier, a colorant, a surfactant, a plasticizer, a lubricant such as magnesium stearate, a flow aid, a compression aid, a solubilizer, an excipient, a diluent such as microcrystalline cellulose, e.g. Avicel PH 102 supplied by FMC corporation, or any combination thereof. Other additives may include phosphate buffer salts, citric acid, glycols, and other dispersing agents. The composition may also include one or more enzyme inhibitors, such as actinonin or epiactinonin and derivatives thereof; aprotinin, Trasylol and Bowman-Birk inhibitor. Further, a transport inhibitor, i.e. a [rho]-glycoprotein such as Ketoprofin, may be present in the compositions of the present disclosure. The solid pharmaceutical compositions of the instant disclosure can be prepared by conventional methods e.g. by blending a mixture of the active compound, the carrier such as 5-CNAC, and any other ingredients, kneading, and filling into capsules or, instead of filling into capsules, molding followed by further tableting or compression-molding to give tablets. In addition, a solid dispersion may be formed by known methods followed by further processing to form a tablet or capsule. Preferably, the ingredients in the pharmaceutical compositions of the instant disclosure are homogeneously or uniformly mixed throughout the solid dosage form.

Alternatively, the active compound may be formulated as a conjugate with said carrier, which may be an oligomer as described in US2003/0069170, e.g.

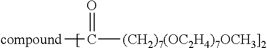

Such conjugates may be administered in combination with a fatty acid and a bile salt as described there.

Conujugates with polyethylene glycol (PEG) may be used, as described for instance in Mansoor et al.

Alternatively, active compounds may be admixed with nitroso-N-acetyl-D,L-penicillamine (SNAP) and Carbopol solution or with taurocholate and Carbapol solution to form a mucoadhesive emulsion.

The active compound may be formulated by loading into chitosan nanocapsules as disclosed in Prego et al (optionally PEG modified as in Prego Prego C, Torres D, Fernandez-Megia E, Novoa-Carballal R, Quiñoá E, Alonso M J.) or chitosan or PEG coated lipid nanoparticles as disclosed in Garcia-Fuentes et al. Chitosan nanoparticles for this purpose may be iminothiolane modified as described in Guggi et al. They may be formulated in water/oil/water emulsions as described in Dogru et al. The bioavailability of active compounds may be increased by the use of taurodeoxycholate or lauroyl carnitine as described in Sinko et al or in Song et al. Generally, suitable nanoparticles as carriers are discussed in de la Fuente et al and may be used in the present disclosure.

Other suitable strategies for oral formulation include the use of a transient permeability enhancer (TPE) system as described in WO2005/094785 of Chiasma Ltd. TPE makes use of an oily suspension of solid hydrophilic particles in a hydrophobic medium to protect the drug molecule from inactivation by the hostile gastrointestinal (GI) environment and at the same time acts on the GI wall to induce permeation of its cargo drug molecules.

Further included is the use of glutathione or compounds containing numerous thiol groups as described in US2008/0200563 to inhibit the action of efflux pumps on the mucous membrane. Practical examples of such techniques are described also in Caliceti, P. Salmaso, S., Walker, G. and Bernkop-Schnurch, A. (2004) ‘Development and in vivo evaluation of an oral insulin-PEG delivery system.’ Eur. J. Pharm. Sci., 22, 315-323, in Guggi, D., Krauland, A. H., and Bernkop-Schnurch, A. (2003) ‘Systemic peptide delivery via the stomach: in vivo evaluation of an oral dosage form for salmon calcitonin’. J. Control. Rel. 92, 125-135, and in Bernkop-Schnurch, A., Pinter, Y., Guggi, D., Kahlbacher, H., Schöffmann, G., Schuh, M., Schmerold, I., Del Curto, M. D., D'Antonio, M., Esposito, P. and Huck, Ch. (2005) ‘The use of thiolated polymers as carrier matrix in oral peptide delivery’—Proof of concept. J. Control. Release, 106, 26-33.

The active compound may be formulated in seamless micro-spheres as described in WO2004/084870 where the active pharmaceutical ingredient is solubilised as an emulsion, microemulsion or suspension formulated into mini-spheres; and variably coated either by conventional or novel coating technologies. The result is an encapsulated drug in “pre-solubilised” form which when administered orally provides for predetermined instant or sustained release of the active drug to specific locations and at specific rates along the gastrointestinal tract. In essence, pre-solubilization of the drug enhances the predictability of its kinetic profile while simultaneously enhancing permeability and drug stability.

One may employ chitosan coated nanocapsules as described in US2009/0074824. The active molecule administered with this technology is protected inside the nanocapsules since they are stable against the action of the gastric fluid. In addition, the mucoadhesive properties of the system enhances the time of adhesion to the intestine walls (it has been verified that there is a delay in the gastrointestinal transit of these systems) facilitating a more effective absorption of the active molecule.

Methods developed by TSR1 Inc. may be used. These include Hydrophilic Solubilization Technology (HST) in which gelatin, a naturally derived collagen extract carrying both positive and negative charges, coats the particles of the active ingredient contained in lecithin micelles and prevents their aggregation or clumping. This results in an improved wettability of hydrophobic drug particles through polar interactions. In addition, the amphiphilic lecithin reduces surface tension between the dissolution fluid and the particle surface.

The active ingredient may be formulated with cucurbiturils as excipients.

Alternatively, one may employ the GIPET technology of Merrion Pharmaceuticals to produce enteric coated tablets containing the active ingredient with an absorption enhancer which may be a medium chain fatty acid or a medium chain fatty acid derivative as described in US2007/0238707 or a membrane translocating peptide as described in U.S. Pat. No. 7,268,214.

One may employ GIRES™ technology which consists of a controlled-release dosage form inside an inflatable pouch, which is placed in a drug capsule for oral administration. Upon dissolution of the capsule, a gas-generating system inflates the pouch in the stomach. In clinical trials the pouch has been shown to be retained in the stomach for 16-24 hours.

Alternatively, the active may be conjugated to a protective modifier that allows it to withstand enzymatic degradation in the stomach and facilitate its absorption. The active may be conjugated covalently with a monodisperse, short-chain methoxy polyethylene glycol glycolipids derivative that is crystallized and lyophilized into the dry active pharmaceutical ingredient after purification. Such methods are described in U.S. Pat. No. 5,438,040 and at www.biocon.com.

One may also employ a hepatic-directed vesicle (HDV) for active delivery. An HDV may consist of liposomes (150 nm diameter) encapsulating the active, which also contain a hepatocyte-targeting molecule in their lipid bilayer. The targeting molecule directs the delivery of the encapsulated active to the liver cells and therefore relatively minute amounts of active are required for effect. Such technology is described in US2009/0087479 and further at www.diasome.com.

The active may be incorporated into a composition containing additionally a substantially non-aqueous hydrophilic medium comprising an alcohol and a cosolvent, in association with a medium chain partial glyceride, optionally in admixture with a long-chain PEG species as described in US2002/0115592 in relation to insulin.

Alternatively, use may be made of intestinal patches as described in Shen Z, Mitragotri S, Pharm Res. 2002 April; 19(4):391-5 ‘Intestinal patches for oral drug delivery’.

The active may be incorporated into an erodible matrix formed from a hydrogel blended with a hydrophobic polymer as described in U.S. Pat. No. 7,189,414.

Suitable oral dosage levels for adult humans to be treated may be in the range of 0.05 to 5 mg, preferably about 0.1 to 2.5 mg.

The frequency of dosage treatment of patients may be from 1 to six times daily, for instance from two to four times daily. Treatment will desirably be maintained over a prolonged period of at least 6 weeks, preferably at least 6 months, preferably at least a year, and optionally for life.

Combination treatments for relevant conditions may be carried out using a composition according to the present disclosure and separate administration of one or more other therapeutics. Alternatively, the composition according to the present disclosure may incorporate one or more other therapeutics for combined administration.

Combination therapies according to the present disclosure include combinations of an active compound as described with insulin, GLP-2, GLP-1, GIP, or amylin, or generally with other anti-diabetics. Thus combination therapies including co-formulations may be made with insulin sensitizers including biguanides such as Metformin, Buformin and Phenformin, TZD's (PPAR) such as Balaglitazone, Pioglitazone, Rivoglitazone, Rosiglitazone and Troglitazone, dual PPAR agonists such as Aleglitazar, Muraglitazar and Tesaglitazar, or secretagogues including sulphonylureas such as Carbutamide, Chloropropamide, Gliclazide, Tolbutamide, Tolazamide, Glipizide, Glibenclamide, Glyburide, Gliquidone, Glyclopyramide and Glimepriride, Meglitinides/glinides (K+) such as Nateglinide, Repaglinide and Mitiglinide, GLP-1 analogs such as Exenatide, Liraglutide and Albiglutide, DPP-4 inhibitors such as Alogliptin, Linagliptin, Saxagliptin, Sitagliptin and Vildagliptin, insulin analogs or special formulations such as (fast acting) Insulin lispro, Insulin aspart, Insulin glulisine, (long acting) Insulin glargine, Insulin detemir), inhalable insulin—Exubra and NPH insulin, and others including alpha-glucosidase inhibitors such as Acarbose, Miglitol and Voglibose, amylin analogues such as Pramlintide, SGLT2 inhibitors such as Dapagliflozin, Remogliflozin and Sergliflozin as well as miscellaneous ones including Benfluorex and Tolrestat.

Further combinations include co-administration or co-formulation with leptins. Leptin resistance is a well-established component of type 2 diabetes; however, injections of leptin have so far failed to improve upon this condition. In contrast, there is evidence supporting that amylin, and thereby molecules with amylin-like abilities, as the salmon calcitonin mimetics, are able to improve leptin sensitivity. Amylin/leptin combination has shown a synergistic effect on body weight and food intake, and also insulin resistance [Kusakabe T et al].

### DESCRIPTION OF THE FIGURES

FIGS. 1A-1B: β-arrestin recruitment dose response by KBP-046-KBP-054 (FIG. 1A) and KBP-058-KBP-063 (FIG. 1B) as a function of calcitonin receptor activation. Dose range is 1 μM to 15 μM and data is shown as fold of vehicle. Screening was conducted in U2OS CALCR Pathhunter cells from DiscoveRx.

FIGS. 2A-2B: β-arrestin recruitment dose response by KBP-046-KBP-054 (FIG. 2A) and KBP-058-KBP-063 (FIG. 2B) as a function of amylin receptor activation. Dose range is 1 μM to 15 μM and data is shown as fold of vehicle. Screening was conducted in CHO-K1 CALCR RAMP3 Pathhunter cells from DiscoveRx.

FIGS. 3A-3B: Prolonged β-arrestin recruitment by KBP-046-KBP-054 (FIG. 3A) and KBP-058-KBP-063 (FIG. 3B) and human calcitonin as a function of calcitonin receptor activation. Single dose of 100 nM at t=0 and after 3, 6, 24, and 72 hours after initial treatment, β-arrestin recruitment was assessed to investigate each ligands protracted potential. Data is shown as fold of vehicle. Screening was conducted in U2OS CALCR Pathhunter cells from DiscoveRx.

FIGS. 4A-43: Protracted attenuation of food intake in SD rats by KBP-046-KBP-054 (FIG. 4A) and KBP-058-KBP-063 (FIG. 4B). After single s.c dose of 2.5 μg/kg of KBP, food intake was monitored in intervals of 0-4 h and 4-24 h to assess the protracted action of individual KBPs. Individual rats were single caged, n=4 in each group.

### EXAMPLES

The presently disclosed embodiments is described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.
The following cell lines expressing the calcitonin, amylin and CGRP receptors were purchased and cultured according to the manufacturer's instructions.
1. Calcitonin Receptor (CTR): U2OS-CALCR from DiscoveRx (Cat. No.: 93-0566C3).
2. Amylin Receptor (AMY-R): CHO-K1 CALCR+RAMP3 from DiscoveRx (Cat. No.: 93-0268C2).
   In independent bioassays, CTR and AMY-R cells were treated with for the indicated timepoints with increasing doses of KBPs identified in Tables 1A and 1B (1000, 250, 62.5, 15.6, 3.9, 1.0, 0.24, 0.06, 0.02 nM and vehicle).

### Example 1: β-Arrestin Assay

PathHunter β-Arrestin GPCR assays are whole cell, functional assays that directly measure the ability of a ligand to activate a GPCR by detecting the interaction of β-Arrestin with the activated GPCR. Because β-arrestin recruitment is independent of G-protein signaling, these assays offer a powerful and universal screening and profiling platform that can be used for virtually any Gi-, Gs, or Gq-coupled receptor.

In this system, the GPCR is fused in frame with the small enzyme fragment ProLink™ and co-expressed in cells stably expressing a fusion protein of β-Arrestin and the larger, N-terminal deletion mutant of β-gal (called enzyme acceptor or EA). Activation of the GPCR stimulates binding of β-Arrestin to the ProLink-tagged GPCR and forces complementation of the two enzyme fragments, resulting in the formation of an active β-gal enzyme. This interaction leads to an increase in enzyme activity that can be measured using chemiluminescent PathHunter® Detection Reagents.

The assay was performed in white 384 well plates (Greiner Bio-One, 784080). Cells were seeded 2500 cells per well in 10 μL cell-type specific medium the day prior to the experiment. To quantify the GPCR-mediated β-arrestin recruitment the Pathhunter™ Detection Kit (93-0001, DiscoverX) was used and assay performed accordingly to the manufacturer's instructions. Furthermore, data from the Pathhunter™ β-arrestin recruitment assay was normalized to an internal standard to improve the distinction between top KBP performers and non-performers.

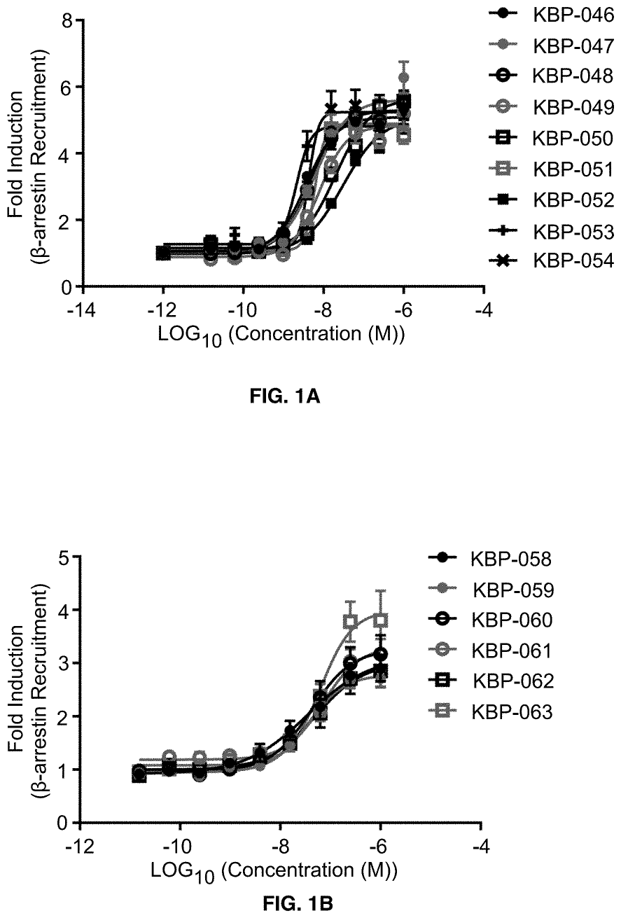

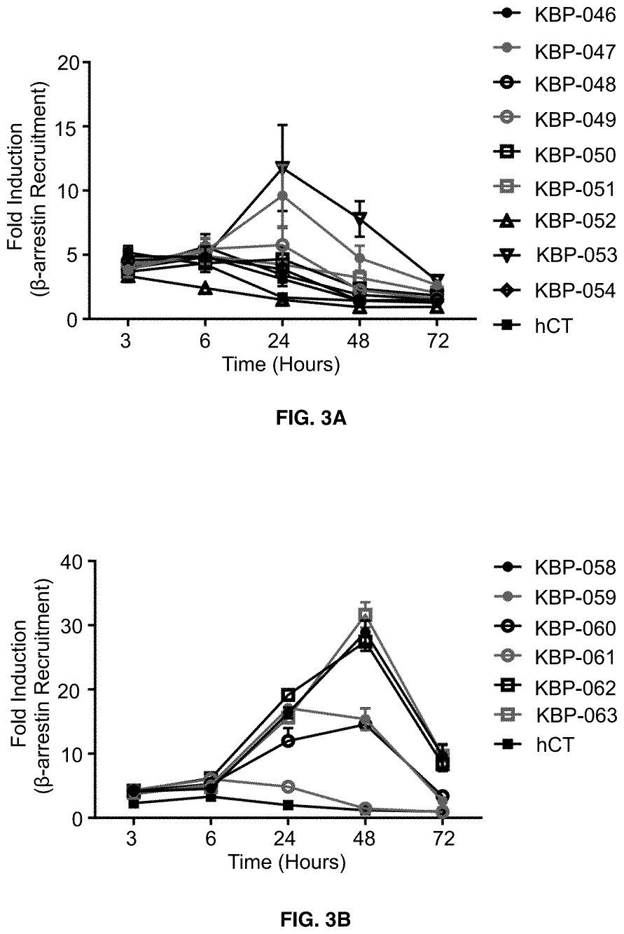

Results are seen in FIGS. 1A-1B, 2A-2B and 3A-3B. As seen in FIGS. 1A-1B, the peptides are quite similar in activity; however, with increasing time KBP-047, KBP-053 (FIG. 3A) and KBP-058, 062 and 063 (FIG. 3B) show a superior ability to activate and maintain activation of the CTR as illustrated in FIG. 3, where the individual ligands are plotted at one given concentration and as a function of time. Here it is apparent that KBP-047, KBP-053, KBP-058, KBP-062 and KBP-063 are superior to the other peptides in terms of protracted calcitonin receptor activation. This was done using the calcitonin receptor (CTR): U2OS-CALCR from DiscoveRx (Cat. No.: 93-0566C3) cell line, and as opposed to the classical three hour output, β-arrestin accumulation was conducted over 24, 48 and 72 hour and then analyzed.

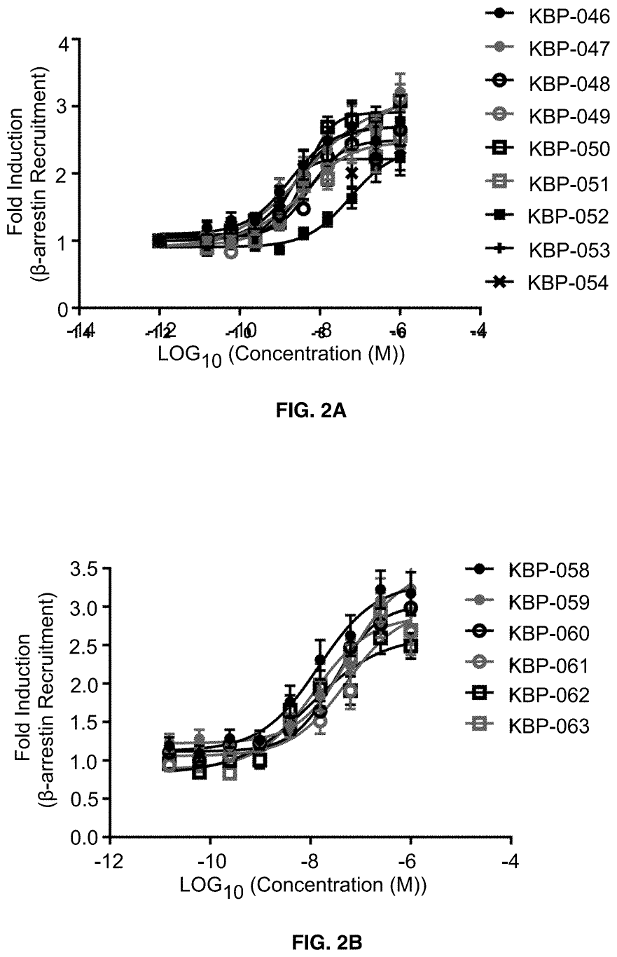

Another important trait of this class of molecules is the ability to activate the amylin receptor. As seen in FIGS. 2A and 2B, the KBPs are fully capable of activating this receptor, and KBP-047, KBP-053, KBP-058, KBP-062 and KBP-063 are demonstrated to be potent amylin agonists. In a subset of the figures, the β-arrestin assay was used to assess prolonged receptor activation.

### Example 2: Comparative Effect of KBP-047 to KBP-063 on Food Intake in Healthy Sprague Dawley Rats

Rats were single caged four days prior to individual tests. They were randomized by weight into seven groups (Vehicle (0.9% NaCl), KBPs (doses: 2.5 μg/kg). They were fasted overnight and then treated with a single dose of peptide or vehicle in the morning using subcutaneous administration. The food intake was monitored in the following intervals (0-4 hours and 4-24 hours).

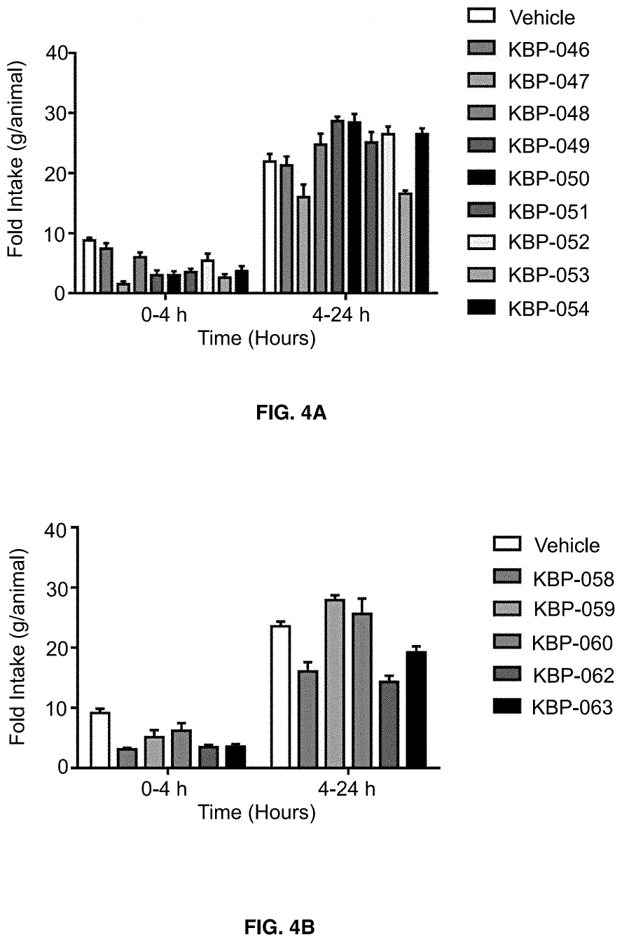

As seen in FIG. 4A, both KBP-047 and KBP-053 led to a reduction in food intake within the 4 hour interval, and the effect was maintained to 24 hours with KBP-047 having the largest reduction, closely followed by KBP-053.

As seen in FIG. 4B, both KBP-058, KBP-062 and KBP-063 led to a reduction in food intake within the 4 hour interval, and the effect was maintained to 24 hours with KBP-062 having the largest reduction, followed by KBP-058 and KBP-063 in that order. An overview of food intake as a function of hCT homology can be found in Table 2.

TABLE 2
In vivo peptide screening characteristics with
corresponding hCT homology percentages.

| | Compound | |
|---|---|---|
| | Ligand Homology | Food Intake |
| | — | ΔFOOD |
| Humanized | Human calcitonin | Sustained |
| KBP Sequences | similarity Percentage | Attenuation Hours (h) |
| KBP-046 | 69% | 4 h |
| KBP-047 | 72% | 24 h |
| KBP-048 | 84% | 4 h |
| KBP-049 | 84% | 4 h |
| KBP-050 | 69% | 4 h |
| KBP-051 | 66% | 4 h |
| KBP-052 | 78% | 4 h |
| KBP-053 | 66% | 24 h |
| KBP-054 | 78% | 4 h |
| KBP-058 | 72% | 24 h |
| KBP-059 | 75% | 4 h |
| KBP-060 | 75% | 4 h |
| KBP-061 | 75% | 4 h |
| KBP-062 | 75% | 24 h |
| KBP-063 | 75% | 24 h |

Overall, KBP-062 has best combination of high hCT homology combined with corresponding top performance in the screening.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference.

1: A peptide, wherein the peptide is:
(SEQ ID NO: 26)
CGNLSTC [symbol] LGRL [symbol] QD [symbol] X4K [symbol] TFP [symbol] TDVGANAP
wherein
X₁=M or V
X₂=T or S
X₃=F or L
X₄=N or H,
X₅=F or L
X₆=Q or H
X₇=Q or K

2: The peptide according to claim 1, wherein X₂ is T, X₃ is L, X₄ is N, X₅ is F, X₆ is H, and/or X₇ is K.

3: A peptide according to claim 1, wherein X₄ is N, X₅ is F, and X₆ is H.

4: The peptide according to claim 1, wherein the peptide has an identity to human calcitonin of at least 65%.

5: The peptide according to claim 1, wherein the peptide is selected from the group consisting of:
(SEQ ID NO: 12)
CGNLSTCMLGRLSQDLNKFHTFPKTDVGANAP,
(SEQ ID NO: 18)
CGNLSTCMLGRLTQDLHKLQTFPKTDVGANAP,
(SEQ ID NO: 20)
CGNLSTCMLGRLTQDFHKLHTFPKTDVGANAP,
(SEQ ID NO: 24)
CGNLSTCMLGRLTQDLNKFHTFPKTDVGANAP,
and [[or]]
(SEQ ID NO: 25)
CGNLSTCMLGRLSQDLNKFHTFPQTDVGANAP

6: The peptide as claimed in claim 1, formulated for enteral administration.

7: The peptide as claimed in claim 1, formulated for parenteral administration.

8: The peptide as claimed in claim 7, formulated for injection.

9: The peptide as claimed in claim 1, formulated with a carrier for oral administration.

10: The peptide as claimed in claim 9, wherein the carrier comprises N-(5-chlorosalicyloyl)-8-aminocaprylic acid (5-CNAC), sodium salt of 10-(2-Hydroxybenzamido)decanoic acid (SNAD), or sodium salt of N-(8-[2-hydroxybenzoyl]amino)caprylic acid (SNAC).

11: A pharmaceutical composition comprising the peptide of claim 6 coated with citric acid particles wherein the coated citric acid particles increase the oral bioavailability of the peptide.

12: (canceled)

13: A method for treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake comprising administering the peptide as claimed in claim 1.

14: The method as claimed in claim 13, comprising administering the peptide in conjunction with metformin or another insulin sensitizer.
